(19) United States
(12) Patent Application Publication
(10) Pub. No.: US 2017/0216177 A1
(43) Pub. Date: Aug. 3, 2017

(54) HAIR REGROWTH TREATMENT AND GROWTH STIMULANT

(71) Applicant: Angelo Perry Thrower, Weston, FL (US)

(72) Inventor: Angelo Perry Thrower, Weston, FL (US)

(21) Appl. No.: 15/341,837

(22) PCT Filed: May 10, 2016
(86) PCT No.: PCT/US16/31589

§ 371 Date: Nov. 2, 2016

Related U.S. Application Data
(60) Provisional application No. 62/163,026, filed on May 18, 2015.

(51) Int. Cl. A61K 8/49; A61K 8/34; A61K 8/97; A61K 8/44; A61K 8/41; A61K 8/55; A61K 36/15; A61K 36/82; A61K 33/30; A61K 31/506; A61K 47/10; A61K 47/44; A61K 47/18; A61K 47/36; A61K 8/73; A61K 47/24; A61K 31/425; A61Q 7/00

(52) U.S. Cl. CPC A61K 8/4953; A61Q 7/00; A61K 8/345; A61K 8/97; A61K 8/44; A61K 8/49; A61K 8/34; A61K 8/416; A61K 8/55; A61K 36/15; A61K 36/82; A61K 33/30; A61K 31/506; A61K 47/10; A61K 47/44; A61K 47/183; A61K 47/36; A61K 8/737; A61K 47/24; A61K 31/425; A61K 2800/74; A61K 2800/596

(57) ABSTRACT

A composition for hair regrowth and hair growth stimulation, a method of use therefore, and a method of making. In an embodiment, the invention is directed toward a hair regrowth and growth stimulant containing the active ingredient minoxidil in combination with a stem cell activating hair growth/regrowth promoter in a non-irritating vehicle/carrier formulation. The non-irritating and scalp-friendly embodiments of the invention are particularly useful in methods for treating all hair types and ethnicities, and are especially useful in the treatment of African-American patients suffering from various forms of non-androgenetic alopecia.

### CROSS-REFERENCE TO RELATED APPLICATION

This application benefit of priority to U.S. Provisional Patent Application Ser. No. 62/163,026, filed May 18, 2015, the contents of which is incorporated herein by reference in its entirety.

### FIELD OF THE INVENTION

This invention generally relates to a composition for hair regrowth and hair growth stimulation, a method of use therefore, and a method of making the composition. The invention more particularly relates to a hair regrowth and growth stimulant containing Minoxidil in a non-irritating carrier formulation useful for all hair types, ethnicities and types of alopecia. The invention most particularly relates to a hair regrowth and growth stimulant formulation particularly formulated to be effective in the treatment of non-androgenetic alopecia which may be experienced by all hair types and ethnicities, but is particularly troublesome for African-American patients suffering therefrom.

### BACKGROUND OF THE INVENTION

Millions of men and women worldwide experience hair thinning or hair loss. The scalp has approximately 110,000 hair follicles, which are growing and falling on a daily basis. When the balance between the growing and the falling hair is altered, hair loss starts and baldness appears.

Androgenetic alopecia or pattern hair loss results from miniaturization of hair follicles in androgen-sensitive areas of the scalp in genetically predisposed persons. Arresting the process of miniaturization remains the goal of medical treatment.

At the present time, topical 6-(piperidin-1-yl)pyrimidine-2,4-diamine 3-oxide, having the common name Minoxidil, and sold under the tradename ROGAINE® by Johnson & Johnson Corporation, and oral finasteride are the only options currently approved by the Food and Drug Administration (USA). In use since 1980, Minoxidil topical solution (MTS) has been a proven treatment for androgenetic alopecia. However, adverse effects have been reported with MTS primarily due to irritation of the scalp, such irritation manifesting as dryness, scaling, itching and redness, which occurs in approximately 7% of patients who use the 2% solution and a greater percentage of those who use the 5% solution because of its higher content of propylene glycol as well as alcohol contents of about 60%. The vehicle in MTS consists of water, alcohol and propylene glycol, of which propylene glycol was found to be the agent most frequently responsible for allergic contact dermatitis. High percentages of alcohol generally tend to exacerbate the irritation by causing scalp dryness. While these adverse effects are prevalent in patient populations exhibiting all hair types and ethnicities, they are particularly prevalent in the African-American population.

While all ethnicities fall victim to androgenetic alopecia, the African-American population is particularly prone to non-androgenetic alopecia. Typically, hair and hair follicles of African-Americans are tightly curled, thus producing hair that spirals. African-American hair also typically has a larger diameter and retains less water, thus it is relatively coarse. Furthermore, the many styling methods utilized on African-American hair cause concern with hair loss. African American hair is very strong, however because the hair cuticle is so thin it is easier for the hair to become damaged with the use of chemicals and tight hair styles.

Most particularly, African-American woman are prone to a variety of hair maladies, specifically traction alopecia, chemical or traumatic alopecia and Central centrifugal cicatricial alopecia (CCCA).

Traction alopecia is a form of hair loss caused by gradual pulling of the hair from tight hairstyles such as ponytails, braids, weaves, dreadlocks, and hair extensions. This type of hair loss is very common among African-American women and is evident by the loss of hair around the temples and the side of the head. Although traction hair loss usually appears in the temporal and parietal areas, it may also be present in the frontal scalp and occipital scalp, as well as the vertex. This is due to prolonged tension from tight braiding or weaving. Traction hair loss also can stem from use of tight rollers, ponytail extensions and aggressive combing or relaxer application. It is also exacerbated by excessive use of relaxers, Brazilian Keratin treatments, texturizers, and other hair chemicals.

Relaxers used to straighten hair can cause a great deal of heat and chemical damage to the hair and scalp, which can also cause Central centrifugal cicatricial alopecia (CCCA), and over time can cause permanent hair loss. Additionally, the use of hot curling irons can lead to traction alopecia. Relaxers, whether with or without lye, have a very high pH. Relaxers break the hair down because they break the bonds that actually give strength to the hair. This causes the hair to straighten. Therefore, relaxed hair is, by definition, weaker than natural hair. Relaxers also deplete the hair of sebum, and this fragile condition, combined with heat from blowdryers and flat irons act in concert to damage the hair and scalp. Hair that has been straightened will be weaker, as opposed to if it were natural, and will be more susceptible to hair breakage (Trichorrhexis Nodosa).

A recently introduced product which appears to show some benefit in hair regrowth is REDENSYL® Hair Regrowth Serum, manufactured by Induchem Corporation. REDENSYL® assists in re-launching stem cell activity and proliferation. The proposed mechanism of action is that the outer root sheath stem cells (ORSc) are vitalized, thus triggering a new hair cycle and increasing the dermal papilla's fibroblast metabolism. Hair follicles are nourished resulting in the stem cells switching on the anagen phase faster.

If a product having the hair regrowth properties of an active ingredient such as Minoxidil could be coupled with a product having the stem cell simulating properties of a product such as REDENSYL®, in a carrier vehicle which was non-irritating and scalp friendly, a long felt need in the treatment of non-androgenetic alopecia would be met.

### DESCRIPTION OF THE PRIOR ART

U.S. Pat. No. 6,482,257 relates to compositions containing i) from about 0.0001% to about 99.9% of certain compounds selected from the group consisting of lupane triterpenes, derivatives of lupane triterpenes, derivatives of oleanane triterpenes, derivatives of ursane triterpenes, and salts and mixtures thereof, and ii) a vehicle.

U.S. Pat. No. 6,596,266, particularly Examples 2,3,4,5 and 9 relates to compositions and/or formulations containing Minoxidil as an active ingredient in combination with other active agents and/or enhancer agents (e.g., saw palmetto extract and nettle root extract). The compositions and/or formulations increase the hair growth capability of the composition. Also disclosed are methods of using the compositions to treat male patterned baldness and to stimulate hair growth on the scalp, including both the apex and frontal regions of the scalp.

U.S. Pat. No. 8,951,507 relates to formulations having an irritation-reducing action, due to inclusion of bisabolol, corresponding cosmetic and pharmaceutical products as well as associated methods and uses thereof.

US Published Patent Application 2012/0114583 relates to a hair treatment agent comprising a combination of dihydroquercetin and/or a dihydroquercetin derivative (a constituent of REDENSYL®) with at least one amino acid. A preferred hair treatment agent disclosed therein comprises a combination of dihydroquercetin (taxifolin) with a six-amino acid mixture consisting of taurine, proline, valine, arginine, lysine, and glycine.

US Published Patent Application 2012/0301527 and International Application WO/11/095970A1 (see Example 9) provides topical pharmaceutical or cosmetic compositions, and uses thereof in treating a disease or condition of the hair follicle. The compositions of this invention are emulsions of an oil-in-polyol with a mean particle size of below one micron, and further comprising at least one oil, one polyol, and one stabilizer.

US Published Patent Application 2012/0283233 relates to a carrier composition comprising a phosphate compound of an electron transfer agent and a polar aprotic solvent. Biologically active compounds formulated with the carrier composition have been shown to have improved properties.

International Application W014/122436A1 relates to a topical pharmaceutical composition comprising Minoxidil and at least one or more pharmaceutically acceptable excipients. Specifically, the reference teaches topical formulations containing Minoxidil, which are essentially devoid of higher amounts of propylene glycol and lower alcohols in order to avoid scalp irritation.

The references fail to teach or suggest a hair regrowth and hair growth stimulating composition which both mitigates miniaturization of hair follicles in androgen-sensitive areas of the scalp and re-launches stem cell activity and proliferation, without substantial scalp irritation, thereby avoiding contact dermatitis of the scalp.

### SUMMARY OF THE INVENTION

The present invention is directed towards a composition for men and women of all hair types and ethnicities worldwide, who experience hair thinning or hair loss. The invention illustrates a product that is effective for providing hair regrowth and hair growth stimulation, a method of use therefore, and a method of making.

In an embodiment, the invention is directed toward a hair regrowth and growth stimulant containing the active ingredient Minoxidil, or a compound functionally equivalent thereto, in a non-irritating vehicle/carrier formulation. Although the non-irritating and scalp-friendly embodiment of the invention is useful for all types of hair and ethnicities, it finds particular utility in methods for treating African-American patients suffering from various forms of non-androgenetic alopecia.

In an embodiment, a stem cell activating composition, illustrated by, albeit not limited to a composition having properties similar to REDENSYL®, is combined with a Minoxidil containing non-irritating vehicle in order to provide a multi-faceted therapeutic composition.

In an embodiment, a process is taught for manufacturing a formulation in accordance with the instant invention.

In an embodiment, a method of application and treatment is taught for treatment of a patient population in need of the formulation in accordance with the present invention.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1A illustrates a view of vertex alopecia prior to treatment;

FIG. 1B illustrates a view of vertex alopecia after treatment;

FIG. 2A illustrates a view of vertex alopecia prior to treatment;

FIG. 2B illustrates a view of vertex alopecia after treatment;

FIG. 3A illustrates a view of traction alopecia prior to treatment;

FIG. 3B illustrates a view of traction alopecia after treatment.

### DETAILED DESCRIPTION OF THE INVENTION

The compositions of the present invention can comprise, consist of, or consist essentially of the essential elements and limitations of the invention described herein, as well any of the additional or optional ingredients, components, or limitations described herein.

The term “comprising” (and its grammatical variations) as used herein is used in the inclusive sense of “having” or “including” and not in the exclusive sense of “consisting only of” The terms “a” and “the” as used herein are understood to encompass the plural as well as the singular.

All percentages, parts and ratios are based upon the total weight of the composition of the present invention, unless otherwise specified. All such weights as they pertain to the listed ingredients are based on the active level and, therefore, do not include earners or byproducts that may be included in commercially available materials, unless otherwise specified.

The term “safe and effective amount” or “therapeutically effective amount” as used herein means an amount of a compound or composition such as a topical or system active sufficient to significantly induce a positive benefit, for example, hair growth or regrowth, but low enough to avoid serious side effects, i.e., to provide a reasonable benefit to risk ratio, within the scope of sound judgment of the skilled artisan.

The topical compositions useful in this invention contain formulations suitable for topical application to skin and scalp. The term “topical” as employed herein relates to the use of a composition along with a suitable pharmaceutical carrier, and applied according to the method of the present invention at the site of hair loss, reduced hair growth or baldness for exertion of local action. Accordingly, such topical compositions useful in the methods of the present invention include those pharmaceutical forms in which the compound is applied externally by direct contact with the skin surface to be treated.

In certain embodiments, a topical pharmaceutical composition according to the instant invention may be in the form of a solution, shampoo, ointment, cream, lotion, emulsion, dispersion, suspension or gel.

In certain embodiments, the compositions according to this invention may further contain one or more additional cosmetically active agent(s) as well as the particularly-mentioned components. What is meant by a “cosmetically acceptable active agent” is a compound, which may be a synthetic compound or a compound isolated, purified or concentrated from a natural source, or a natural extract containing a mixture of compounds, that has a cosmetic effect on the tissue, including, but not limited to: anti-aging agents, sunscreens, photoprotectors, antioxidants, keratolytic agents, detergents/surfactants, moisturizers, nutrients, vitamins, minerals, energy enhancers, anti-perspiration agents, astringents, hair growth enhancing agents, hair coloring agents, pigments, firming agents, agents for skin conditioning, and odor-control agents such as odor masking or pH-changing and buffering agents.

In certain embodiments, the cosmetically acceptable active is a hair growth active, including, albeit not limited to compounds known to promote hair growth and available as drugs, such as diazoxide, pinacidil, bimatoprost, finasteride, a type 2 5-alpha-reductase inhibitor, and dutasteride, a type 1- and 2-5-alpha-reductase inhibitor, as well as flutamide, bicalutamide, pregnane derivatives, progesterone derivatives, experimental agents such as FCE 28260 and the like. Spironolactone and other diuretics may also be utilized as it is indicated for women in some cases (also known as aldactone: an aldosterone receptor antagonist).

The topical compositions or topical pharmaceutical compositions useful in this invention contain formulations suitable for topical application to skin and scalp. The term “topical” as employed herein relates to the use of a composition along with a suitable pharmaceutical carrier, and applied according to the method of the present invention at the site of hair loss, reduced hair growth or baldness for exertion of local action. Accordingly, such topical compositions useful in the methods of the present invention include those pharmaceutical forms in which the compound is applied externally by direct contact with the skin surface to be treated.

Suitable carrier forms include ointments, pastes, gels, jellies, serums, aerosol and non-aerosol sprays, foams, creams, lotions, solutions, suspensions and the like. The term “ointment” embraces formulations (including creams) having oleaginous, absorption, water-soluble and emulsion-type bases, e.g., petrolatum, lanolin, polyethylene glycols, as well as mixtures of these.

Various other materials may also be present in the compositions useful in the subject invention. These include any manner of humectants, proteins and polypeptides, an alkaline agent and mixtures thereof. The compositions of the present invention may also comprise one or more of various additives and excipients including plant extracts, oily ingredients, surfactants, alcohols, fatty acids, preservatives, antioxidants, colorants, fragrances, UV absorbers, viscosity modifiers, chelating agents(e.g., EDTA), pH-adjusting agents, and vitamins. In addition, the topical compositions useful herein can contain conventional 15 cosmetic adjuvants, such as dyes, sunscreen (e.g., titanium dioxide), pigments, and fragrances.

In a particular embodiment, the pharmaceutically acceptable excipients may include one or more solvents, one or more surfactants, one or more penetration enhancers, one or more pH regulators, one or more preservatives, one or more viscosity modifiers, one or more anti-inflammatory agents, one or more moisturizers, one or more conditioning agents, one or more colorants, one or more fragrances, and one or more chelating agents.

In one or more embodiments of the present disclosure, illustrative, albeit non-limiting examples of chelating agents may be at least one of ethylenediaminetetraacetic acid (“EDTA”) and salts thereof such as disodium EDTA, tetrasodium EDTA and calcium disodium EDTA; diethylenetriammepentaacetic acid (“DTPA”) and salts thereof; hydroxyethlethylenediaminetriacetic acid (“HEDTA”) and salts thereof and nitrilotriacetic acid (“NTA”); more preferably EDTA, HEDTA and their salts; most preferably EDTA and its saltsacetyl trihexyl citrate, aminotrimethylene phosphonic acid, beta-alanine diacetic acid, bismuth citrate, citric acid, cyclohexanediamine tetraacetic acid, diammonium citrate, dibutyl oxalate, diethyl oxalate, diisobutyl oxalate, diisopropyl oxalate, dilithium oxalate, dimethyl oxalate, dipotassium oxalate, dipropyl oxalate, disodium pyrophosphate, etidronic acid, methyl cyclodextrin, oxalic acid, pentapotassium, triphosphate, pentasodium aminotrimethylene phosphonate, pentasodium pentetate, pentasodium triphosphate, pentetic acid, phytic acid, potassium citrate, sodium citrate, sodium dihydroxyethylglycinate, sodium gluceptate, sodium gluconate, sodium hexametaphosphate, sodium metaphosphate, sodium metasilicate, sodium oxalate, sodium trimetaphosphate, tetrahydroxypropyl ethylenediamine, tetrapotassium etidronate, tetrapotassium pyrophosphate, tetrasodium etidronate, tetrasodium pyrophosphate, trisodium NTA, trisodium phosphate, malic acid, fumaric acid, maltol, succimer, penicillamine, dimercaprol, and desferrioxamine mesilate.

In one or more embodiments of the present disclosure, illustrative, albeit non-limiting examples of moisturizing agents may be at least one of amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol, acetylated lanolin, acetylated lanolin alcohol, acrylates/C₁₀₋₃₀ alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, camauba (copemicia cerifera) wax, carrageenan (chondrus crispus), carrot (daucus carota sativa) oil, castor (ricinus communis) oil, ceramides, ceresin, ceteareth-5, ceteareth-12, ceteareth-20, 10 cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceiyl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesqui stearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxy stearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C₁₂₋₁₈ ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesqui stearate, PEG40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquatemium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quatemium-15, quatemium-18 hectorite, quatemium-22, retinol, retinol palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, semm protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

In one or more embodiments of the present disclosure, illustrative, albeit non-limiting examples of thickening agents (viscosity modifiers) may be at least one of thickener or gelling agents, including substances that can increase the viscosity of a composition. Thickeners include those that can increase the viscosity of a composition without substantially modifying the efficacy of the active ingredient within the composition. Thickeners can also increase the stability of the compositions of the present invention. Non-limiting examples of thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted aciylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053, CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose 10 or pentaerytritol (e.g., CARBOPOL™ 900 series from B. F. Goodrich).

In one or more embodiments of the present disclosure, illustrative, albeit non-limiting examples of preservatives may be at least one of benzoic acid, the esters and salts thereof, propionic acid and salts thereof, salicylic acid and salts thereof, 2,4-hexanoic acid (sorbic acid) and salts thereof, formaldehyde and paraformaldehyde, 2-hydroxybiphenyl ether and salts thereof, 2-zincsulphidopyridine-N-oxide, inorganic sulphites and bisulphites, sodium iodate, chlorobutanolum, 4-hydroxybenzoic acid, the salts and esters thereof, dehydroacetic acid, formic acid, 1,6-bis(4-amidino-2-bromophenoxy)-n-hexane and salts thereof, the sodium salt of ethylmercury-(II)-thiosalicylic acid, phenyl mercury and salts thereof, 10-undecylenic acid and salts thereof, 5-amino-1,3-bis(2-ethylhexyl)-5-methylhexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)urea, 4-chloro-m-cresol, 2,4,4′-trichloro-2′-bydroxy-diphenyl ether, 4-chloro-3,5-dimethylphenol, 1,1′-methylene-bis(3-(1-hydroxymethyl-2,4-dioximidazolidin-5-yl)urea), poly-(hexamethylene diguanide) hydrochloride, 2-phenoxyethanol, hexamethylentetramine, 1-(3-chloroallyl)-3,5,7-triaza-1-azonia-adamantane chloride, 1 (4-chloro-phenoxy)-1(1H-imidazol-1-yl)-3,3-dimethyl-2-butanone, 1,3-bis-(hydroxy-inethyl)-5,5-dimethyl-2,4-imidazolidinedione, benzyl alcohol, Octopirox, 1,2-dibromo-2,4-dicyanobutane, 2,2′-methylene-bis(6-bromo-4-chloro-phenol), bromochlorophene, mixture of 5-chloro-2-methyl-3(2H)-isothiazolinone and 2-methyl-3(2H)isothiazolinone with magnesium chloride and magnesium nitrate, 2-benzyl-4-chlorophenol, 2-chloracetamide, chlorhexidine, chlorhexidine acetate, chlorhexidine gluconate, chlorhexidine hydrochloride, 1-phenoxy-propan-2-ol, N-alkyl(C₁₂-C₂₂) trimethylammonium bromide and chloride, 4,4-dimethyl-1,3-oxazolidine, N-hydroxymethyl-N-(1,3-di(hydroxymethyl)-2,5-dioxo-imidazolidin-4-yl)-N′—hydroxymethylurea, 1,6-bis(4-amidino-phenoxy)-n-hexane and salts thereof, glutaraldehyde 5-ethyl-1-aza-3,7-dioxa-bicyclo(3.3.0)octane, 3-(4-chlorphenoxy)-I,2-propanediol, hyamine, alkyl-(C₈-C₁₈)-dimethylbenzylammonium chloride, alkyl-(C₈-C₁₈)-dimethylbenzylammonium bromide, alkyl-(C₈-C₁₈)-dimethyl-benzyl-ammonium saccharinate, benzylhemiformal, 3-iodo-2-propinylbutyl carbamate, sodium hydroxymethylaminoacetate or sodium hydroxy-methylaminoacetate.

Minoxidil (i.e. 2,4-diamino-6-piperidinylpyrimidine-3-oxide) is a well-known pharmaceutical compound. It is marketed by The Upjohn Company as the active ingredient in LONITEN® tablets for the treatment of hypertension. It is also useful in topical compositions for the treatment of baldness and is sold under the tradename ROGAINE® by Johnson & Johnson Corporation. The structure and use of this compound for this purpose, and topical compositions containing it, are described in U.S. Pat. No. 4,139,619 and U.S. Pat. No. 4,596,812. In particular, U.S. Pat. No. 4,139,619 discloses topical Minoxidil compositions containing carriers selected from ointments, lotions, pastes, jellies, sprays and aerosols.

It is within the purview of this invention to further utilize, either alone, or in combination, compounds which are similar in structure and function to Minoxidil, exemplified by, albeit not limited to PDPO (Pyrrolidinyl Diaminopyrimidine Oxide—trade name Triaminodil™) and diaminopyrimidine oxide, trade name Aminexil.

It is further within the purview of this invention to utilize minoxidil in the form of a pharmaceutically acceptable derivative which optionally comprises pharmaceutically acceptable salts, solvates, hydrates, isomers, esters, tautomers, anhydrates, enantiomers, complexes, polymorphs or prodrugs.

It is noted that products such as liquid ROGAIN® (a commercially available Minoxidil containing scalp treatment for both men and women) contains about 60% alcohol and ROGAINE® foam contains about 30% alcohol, while the formulation of the instant invention only contains about 20% alcohol.

### EXAMPLE I

In one illustrative, albeit non-limiting embodiment of the invention, the active ingredient Minoxidil—at about 5% (wt/wt) is combined with the following additional ingredients (all ingredients are % (wt/wt)):
about 30% (wt/wt) Propylene Glycol;
about 15% (wt/wt) Isopropyl Alcohol (Anhydrous);
about 9.00% (wt/wt)Dimethyl Isosorbide (ARLASOLYE DMI);
about 3.00% (wt/wt) Trimethylpentanediol/Adipic Acid Copolymer (LEXOREZ TL-8);
about 3.50% (wt/wt) Cetearyl Alcohol, Dicetyl Phosphate, and Ceteth-10 Phosphate (CRODAFOS CES-PA);
about 3.0% (wt/wt) of a stem cell activating hair growth/regrowth promoter exemplified by, albeit not limited to REDENSYL” hair growth promoter, which is an aqueous composition including about 50-55% (wt/wt) Glycerin, about 1% (wt/wt) Larix Europaea Wood Extract, about 1% (wt/wt) Glycine, about 0.1% (wt/wt) Zinc Chloride, about 0.1% (wt/wt) Camellia 5 Sinensis Leaf Extract and 45-50% (wt/wt) purified water;
about 1.4% (wt/wt) Cetyl Alcohol;
about 0.7% (wt/wt) Argania spinosa (Argan) Nut Oil;
about 0.28% (wt/wt) Perfume (e.g. Fragrance79734);
about 0.07% (wt/wt) Cocos Nucifera Oil/Aloe Barbadensis Leaf Extract—(Aloe Butter); about 0.525% (wtlwt) Cetrimonium Chloride;
about 0.2% (wtlwt) Disodium EDTA;
about 0.7% (wtlwt) Bisabolol;
about 0.07% (wt/wt)Hydroxypropyl Guar (N-HANCE HP-40);
Citric Acid in an amount sufficient to maintain pH in range of 6.4-6.6;
about 0.0175 Methylchloroisothiazolinone, Methylisothiazolinone—(KATHON CG);
approximately 0.0001-0.0002 FD&C Yellow #6—(to achieve desired coloration); and purified water to dilute to 100% (wt/wt).

In this illustrative embodiment, the formulation may be manufactured in accordance with the following steps:

### Part A

Add into the main tank A the following ingredients one at a time and mix well until completely dissolved.

### DI Water to 100

### Disodium EDTA 0.20

### FD&C Yellow #6 q.c.

Mix for 15 minutes. Add the following ingredients and mix until completely hydrated.

### N-HANCE® HP40 0.07

Start to heat the main tank to 175° F. while mixing.

### Part B

Add all ingredients from part B. Start to heat the tank to 175° F. while mixing until all ingredients are melted.

### Crodafos CES-PA 3.50

### Cetyl Alcohol 1.40

### Aloe Butter 0.07

### Argan Oil 0.70

Keep mixing with moderate speed for 45 minutes.

### Part C

Start to cool down the tank to 104° F. and add the ingredients from Part C to the tank one at a time while mixing after each ingredient.

### Centrimonium Chloride 0.525

### KATHON®CG 0.0175

### Fragrance 79/34 0.28

### Part D

Keep mixing until uniform smooth cream.

### Part E

In a separate container dissolve Minoxidil in Propylene Glycol and IPA (Isopropyl alcohol).

### Minoxidil 5.00

### Propylene Glycol 30.00

### ARLASOLVE® DMI 9.00

### IPA Anhydrous 15.00

### LEXOREZ® TL-8 3.00

### Part F

Add conditioner into Part E and mix well until the cream becomes uniform again. Use homogenizer if needed.

### Part G

Add the following ingredients and mix well until uniform.

### REDENSYL® 3.00

### Citric Acid q.s.

### Typical Properties

### pH: 4.5-5.0 Viscosity: TBDcPs @20 RPM Spindle 5

### EXAMPLE 2

In another illustrative, albeit non-limiting embodiment of the invention, the active ingredient Minoxidil—at about 5% (wt/wt) is combined with the following additional ingredients (all ingredients are % (wt/wt)):
about 50% (wt/wt) Propylene Glycol;
about 20% (wt/wt) Ethanol (SDA 40/200 proof);
about 3.00% (wt/wt) Trimethylpentanediol/Adipic Acid Copolymer (LEXOREZ TL-8);
about 2.40% (wt/wt) Cetearyl Alcohol, Dicetyl Phosphate, and Ceteth-10 Phosphate (CRODAFOS CES-PA);
about 3.0% (wt/wt) of a stem cell activating hair growth/regrowth promoter exemplified by, albeit not limited to REDENSYL® hair growth promote, which is an aqueous composition including about 50-55% (wt/wt) Glycerin, about 1% (wt/wt) Larix Europaea Wood Extract, about 1% (wt/wt) Glycine, about 0.1% (wt/wt) Zinc Chloride, about 0.1% (wt/wt) Camellia Sinensis Leaf Extract and 45-50% (wt/wt) purified water—
about 0.8% (wt/wt) Cetyl Alcohol;
about 0.2% (wt/wt) Argania spinose (Argan) Nut Oil;
about 0.14% (wt/wt) Perfume (e.g. Fragrance 79734);
about 0.20% (wt/wt) Cocos Nucifera Oil/Aloe Barbadensis Leaf Extract—(Aloe Butter);
about 0.30% (wt/wt) Cetrimonium Chloride;
about 0.2% (wt/wt) Disodium EDTA;
about 0.2% (wt/wt) Bisabolol;
about 0.04% (wt/wt) Hydroxypropyl Guar (N-HANCE HP-40);
Citric Acid in an amount sufficient to maintain pH in range of 6.4-6.6;
about 0.01 Methylchloroisothiazolinone, Methylisothiazolinone—(KATHON CG);
approximately 0.0001-0.0002 FD&C Yellow #6—(to achieve desired coloration); and
purified water to dilute to 100% (wt/wt).

In this illustrative embodiment, the formulation may be manufactured in accordance with the following the steps:

### Part A

Add into the main tank A the following ingredients one at a time and mix well until completely dissolved.

### DI Water to 100

### Disodium EDTA 0.20

### FD&C Yellow #6 q.c.

Mix for 15 minuies. Add the following ingredient and mix until completely hydrate.

### N-HANCE HP-40 0.04

Start to heat the main tank to 175° F. while mixing.

### Part B

Add all ingredients from part B. Start to heat the tank to 175° F. while mixing until all ingredients are melted.

### Crodafos CES-PA 2.40

### Cetyl Alcohol 0.80

### Aloe Butter 0.20

### Argan Oil 0.20

Keep mixing with moderate speed for 45 minutes.

### Part C

Start to cool down the tank to 104° F. and add the ingredients from Part C to the tank one at a time while mixing after each ingredient.

### Cetrimonium Chloride 0.30

### Kathon CG 0.01

### Fragrance 79734 0.14

### Bisabolol 0.20

### Part D

Keep mixing until uniform smooth cream.

### Part E

In a separate container dissolve Minoxidil in Propylene Glycol and IPA.

### Minoxidil 5.00

### Propylene Glycol 50.00

SDA 40/200 proof 20.00

### Lexorez TL-8 3.00

### Part F

Add conditioner into Part E and mix well until cream is uniform again. Use homogenizer if needed.

### Part G

Add the following ingredient and mix until uniform.

### Redensyl 3.00

### Citric Acid q.s.

### Typical Properties

### pH:4.5-5.0 Viscosity: TBD cPs @20 RPM Spindle 5

Specifically recited ingredients are commercially available as follows:

| TRADE NAME | INCI | MANF. | ACTION |
|---|---|---|---|
| DI Water | Aqua | | Solvent |
| Disodium EDTA | Di sodium | Prochem | Chelating Agent |
| | EDTA | | |
| N-HANCE ® HP40 | Hydroxypropyl | Ashland Specialties | Conditioner |
| | Guar | | |
| Argan Oil | Argania spinosa | CAC Farpint | Moisturizer, Regenerate |
| | (Argan) Nut Oil | | |
| Crodafos CES-PA | Cetearyl Alcohol | Croda | Conditioning Agent |
| | (and) Dicetyl | | |
| | Phosphate (and) | | |
| | Ceteth-10 | | |
| | Phosphate | | |
| Cetyl Alcohol | Cetyl Alcohol | Lipo | Viscosity Modifier |
| | | Chemicals/Protameen | |
| Aloe Butter | Cocos Nucifera | Flail Star | Moisturizer |
| | Oil/Aloe | | |
| | Barbadensis | | |
| | Leaf Extract | | |
| Cetrimonium | Cetrimonium | Essential Ingredients | Conditioning Agent |
| Chloride | Chloride | | |
| KATHON ® CG | Methyl chloro | Rohm&Haas; Dow | Preservative |
| | isothiazolinone; | Chemicals | |
| | Methyl | | |
| | isothiazolinone | | |
| Fragrance 79734 | Fragrance | Belle-Aire Fragrances | Aroma |
| FD&C Yellow #6 | FD&C Yellow | Essential Ingredients | Colorant |
| | #6 | | |
| LEXOREZ ® TL-8 | Trimethylpentanediol/ | Inolex | Delivery System |
| | Adipic | | |
| | Acid | | |
| | Copolymer | | |
| Minoxidil | Minoxidil | Letco Medical | Hair Growth |
| REDENSYL ® | Glycerin (and) | Induchem USA | Hair Growth |
| | Aqua (and) | | |
| | Sodium | | |
| | Metabisulfite | | |
| | (and) Larix | | |
| | Europaea Wood | | |
| | Extract (and) | | |
| | Glycine (and) | | |
| | Zinc Chloride | | |
| | (and) Camellia | | |
| | Sinensis Leaf | | |
| | Extract | | |
| Bisabolol | Bisabolol | BASF | Anti-inflammatory |
| ARLASOLVE ® | Dimethyl | Croda | Solvent, Delivery for |
| DMI | Isosorbide | | actives |
| Citric Acid | Citric Acid | Prochem | Chelating Agent |
| SDA40/200 Proof | Ethanol | Univar | Solvent |
| IPA Isopropyl | Isopropanol | Sigma-Aldrich | Solvent |
| Alcohol | | | |

In an illustrative embodiment of the instant invention, the formulation is provided for use in a bottle pump. An illustrative method of treatment is outlined in the following steps:
With the bottle pump, apply one pump (approximately 1 mL) of the formulation two 5 times a day (once in the morning and one hour before bedtime) to the scalp in the hair loss area.
Massage into scalp with fingers, then wash hands well.
Continue daily use in order to maintain hair regrowth and mitigate continued hair loss.

Patients generally begin to see results in about four months. Up to eight months of usage may be needed to achieve the best results. Those patients who achieve a positive response will need to continue to use the formulation two times a day to keep and continue the hair regrowth.

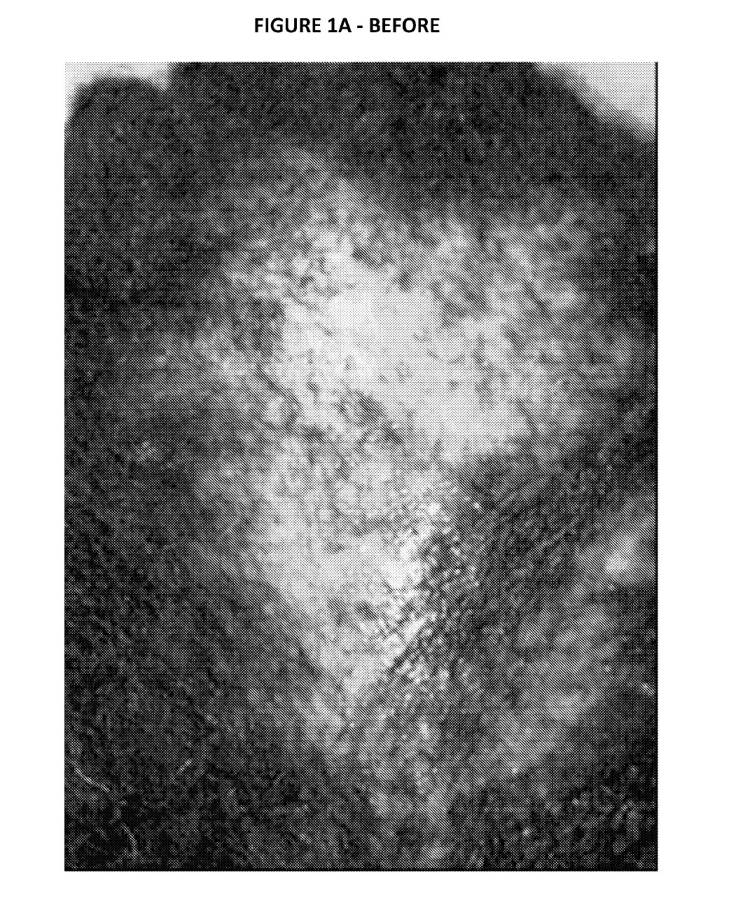

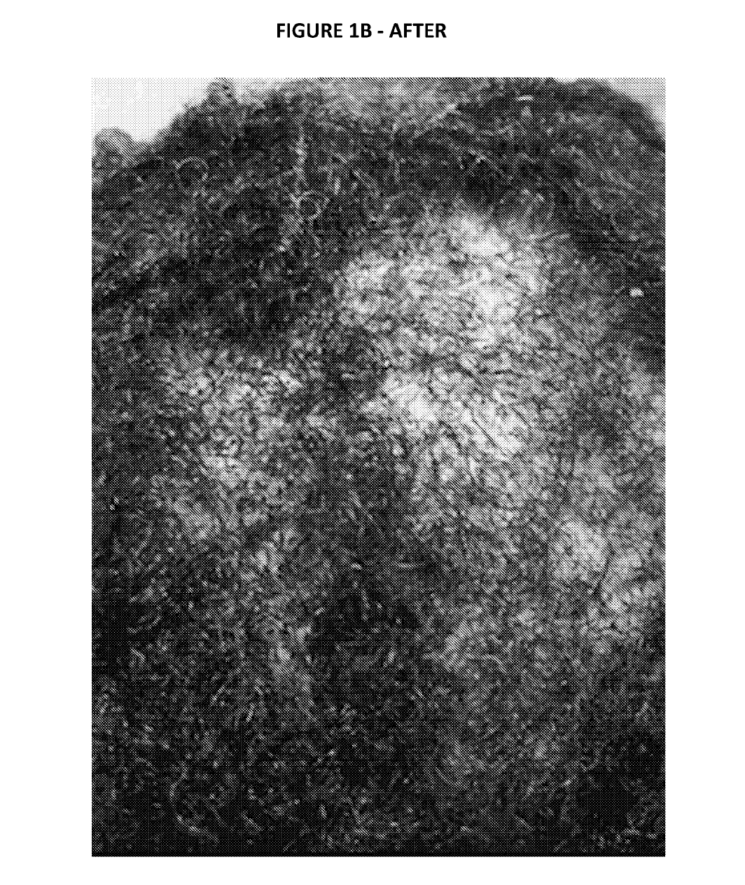

FIG. 1A illustrates a view of vertex alopecia prior to treatment;

FIG. 1B illustrates a view of vertex alopecia after 4-6 months of treatment;

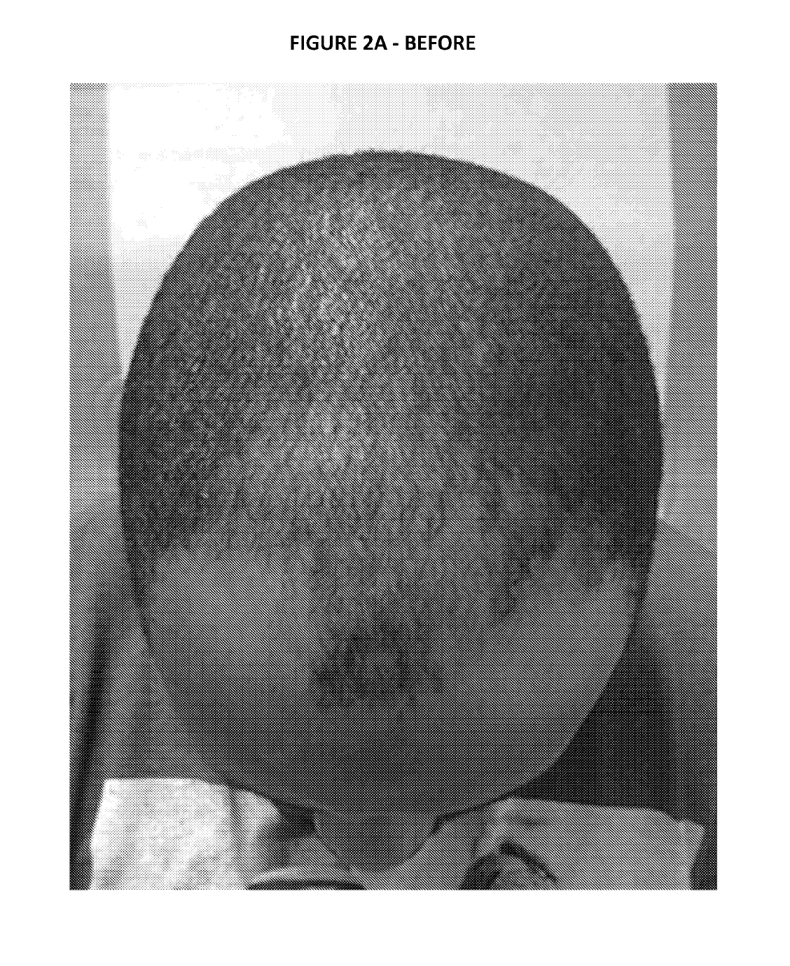

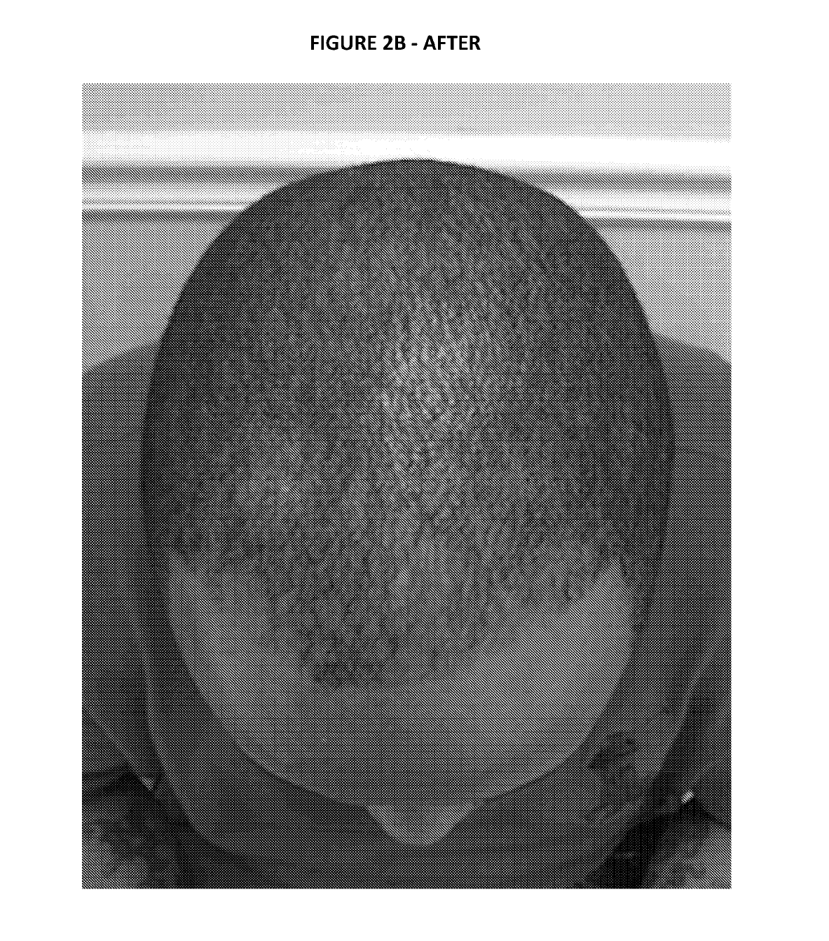

FIG. 2A illustrates a view of vertex alopecia prior to treatment;

FIG. 2B illustrates a view of vertex alopecia after 4-6 months of treatment;

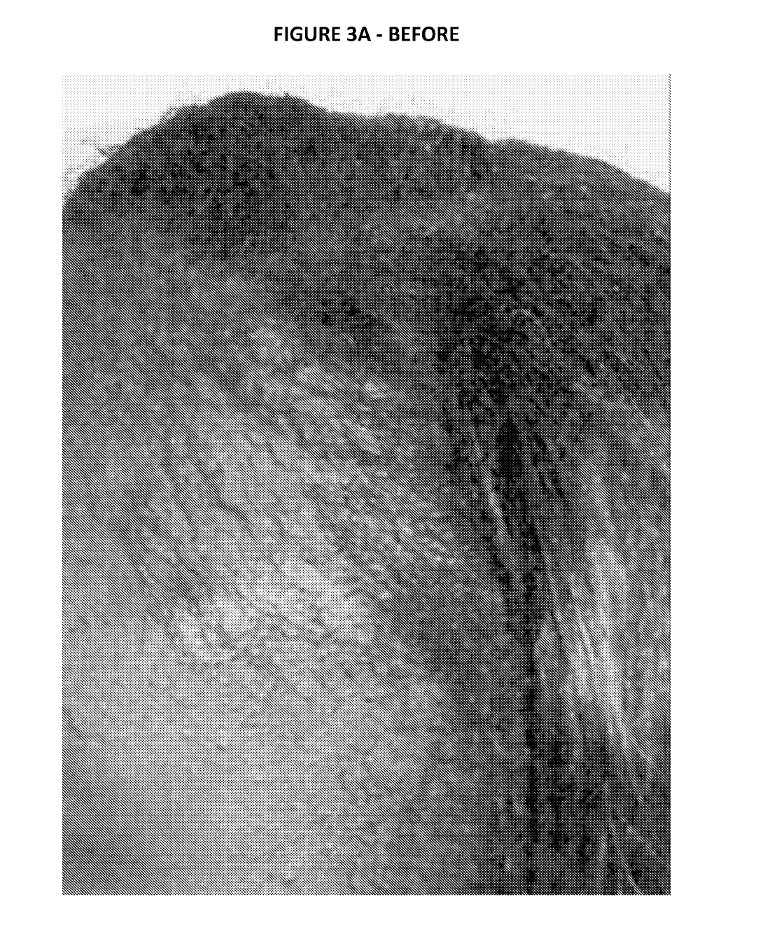

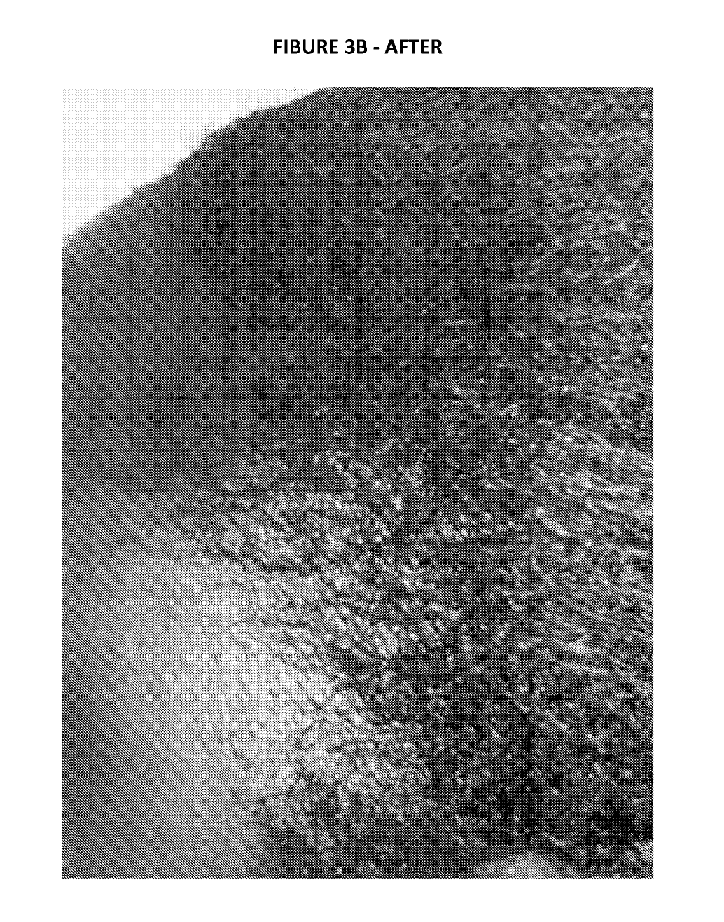

FIG. 3A illustrates a view of traction alopecia prior to treatment;

FIG. 3B illustrates a view of traction alopecia after 4-6 months of treatment.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A topical pharmaceutical composition comprising a therapeutic amount of minoxidil or a pharmaceutically acceptable derivative thereof, in combination with a therapeutic amount of a stem cell activating hair growth/regrowth promoter, which comprises an aqueous composition including about 50-55% (wt/wt) Glycerin, about 1% (wt/wt) Larix Europaea Wood Extract, about 1% (wt/wt) Glycine, about 0.1% (wt/wt) Zinc Chloride, about 0.1% (wt/wt) Camellia Sinensis Leaf Extract and 45-50% (wt/wt) purified water.

2. The composition of claim 1 wherein said pharmaceutically acceptable derivative of minoxidil may comprise one or more pharmaceutically acceptable salts, solvates, hydrates, isomers, esters, tautomers, anhydrates, enantiomers, complexes, polymorphs or prodrugs.

3. The composition of claim 1, further including one or more pharmaceutically acceptable excipients, including one or more solvents, one or more surfactants, one or more penetration enhancers, one or more pH regulators, one or more preservatives, one or more viscosity modifiers, one or more anti-inflammatory agents, one or more moisturizers, one or more conditioning agents, one or more colorants, one or more fragrances, and one or more chelating agents.

4. A topical pharmaceutical composition comprising:
a) a therapeutic amount of minoxidil or a pharmaceutically acceptable derivative thereof;
b) a therapeutic amount of a stem cell activating hair growth/regrowth promoter,
which is an aqueous composition including about 50-55% (wt/wt) Glycerin, about 1′% (wt/wt) Larix Europaea Wood Extract, about 1% (wt/wt) Glycine, about 0.1% (wt/wt) Zinc Chloride, about 0.1% (wt/wt) Camellia Sinensis Leaf Extract and 45-50% (wt/wt) purified water; and
c) a pharmaceutically acceptable carrier,
wherein the composition is effective to treat alopecia.

5. The topical pharmaceutical composition in accordance with claim 4, wherein the minoxidil or a pharmaceutically acceptable derivative thereof is present in an amount of about 5% (wt/wt).

6. The topical pharmaceutical composition in accordance with claim 4, wherein the stem cell activating hair growth/regrowth promoter is present in an amount of about 3% (wt/wt).

7. The topical pharmaceutical composition in accordance with claim 4, further including an alcohol solvent selected from the group consisting of ethanol and isopropanol.

8. The topical pharmaceutical composition according to claim 1, wherein said composition is in the form of a solution, shampoo, ointment, cream, lotion, emulsion, dispersion, suspension or gel.

9. A topical pharmaceutical composition comprising:
about 5% (wt/wt) minoxidil, or a pharmaceutically acceptable derivative thereof;
about 30% (wt/wt) Propylene Glycol;
about 15% (wt/wt) Isopropyl Alcohol (Anhydrous);
about 9.00% (wt/wt) Dimethyl Isosorbide;
about 3.00% (wt/wt) Trimethylpentanediol/Adipic Acid Copolymer;
about 3.50% (wt/wt) Cetearyl Alcohol, Dicetyl Phosphate, and Ceteth-10 Phosphate;
about 3.0% (wt/wt) of a stem cell activating hair growth/regrowth promoter, which is an aqueous composition comprising about 50-55% (wt/wt) Glycerin, about 1% (wt/wt) Larix Europaea Wood Extract, about 1% (wt/wt) Glycine, about 0.1% (wt/wt) Zinc Chloride, about 0.1% (wt/wt) Camellia Sinensis Leaf Extract and 45-50% (wt/wt) purified water;
about 1.4% (wt/wt) Cetyl Alcohol;
about 0.7% (wt/wt) Argania spinosa (Argan) Nut Oil;
about 0.28% (wt/wt) fragrance;
about 0.07% (wt/wt) Cocos Nucifera Oil/Aloe Barbadensis Leaf Extract;
about 0.525% (wt/wt) Cetrimonium Chloride;
about 0.2% (wt/wt) Disodium EDTA;
about 0.7% (wt/wt) Bisabolol;
about 0.07% (wt/wt) Hydroxypropyl Guar;
Citric Acid in an amount sufficient to maintain pH in a range of 6.4-6.6;
about 0.0175 kg/lb Methylchloroisothiazolinone, Methylisothiazolinone;
approximately 0.0001-0.0002 kg/lb of a colorant; and
purified water to dilute to 100% (wt/wt).

10. A topical pharmaceutical composition comprising:
about 5% (wt/wt) minoxidil, or a pharmaceutically acceptable derivative thereof;
about 50% (wt/wt) Propylene Glycol;
about 20% (wt/wt) Ethanol;
about 3.00% (wt/wt) Trimethylpentanediol/Adipic Acid Copolymer;
about 2.40% (wt/wt) Cetearyl Alcohol, Dicetyl Phosphate, and Ceteth-10 Phosphate;
about 3.0% (wt/wt) of a stem cell activating hair growth/regrowth promoter, which comprises an aqueous composition including about 50-55% (wt/wt) Glycerin, about 1% (wt/wt) Larix Europaea Wood Extract, about 1% (wt/wt) Glycine, about 0.1% (wt/wt) Zinc Chloride, about 0.1% (wt/wt) Camellia Sinensis Leaf Extract and 45-50% (wt/wt) purified water;
about 0.8% (wt/wt) Cetyl Alcohol;
about 0.2% (wt/wt) Argania spinosa (Argan) Nut Oil;
about 0.14% (wt/wt) fragrance;
about 0.20% (wt/wt) Cocos Nucifera Oil/Aloe Barbadensis Leaf Extract;
about 0.30% (wt/wt) Cetrimonium Chloride;
about 0.2% (wt/wt) Disodium EDTA;
about 0.2% (wt/wt) Bisabolol;
about 0.04% (wt/wt) Hydroxy propyl Guar;
Citric Acid in an amount sufficient to maintain pH in a range of 6.4-6.6;
about 0.01 kg/lb of a mixture of Methylchloroisothiazolinone and Methylisothiazolinone; approximately 0.0001-0.0002 kg/lb of a colorant; and purified water to dilute to 100% (wt/wt).

11. (canceled)

12. A process for preparing the topical pharmaceutical composition according to claim 1, wherein the process comprises blending minoxidil or a pharmaceutically acceptable derivative thereof, in combination with a stem cell activating hair growth/regrowth promoter, with at least one pharmaceutically acceptable excipient.

13. The topical pharmaceutical composition according to claim 1, wherein the alopecia is a non-androgenetic alopecia.

14. The topical pharmaceutical composition according to claim 1, wherein the non-androgenetic alopecia is selected from the group consisting of vertex alopecia, chemical alopecia, traction alopecia, traumatic alopecia or central centrifugal cicatricial alopecia (CCCA).

15. The topical pharmaceutical composition according to claim 1, wherein the subject is an Afro-American human subject.

16. The topical pharmaceutical composition according to claim 1, wherein the composition is effective to mitigate miniaturization of hair follicles in androgen-sensitive areas of the scalp, and to re-launch stem cell activity and proliferation, without substantial scalp irritation.

17. A method for treating alopecia comprising administering topically a therapeutic amount of the composition of claim 1, wherein the therapeutic amount of the composition is effective:
(a) to induce hair growth; or
(b) to stimulate hair growth; or
(c) to reduce hair loss; or
(d) to reduce scalp irritation; or
(e) to reduce contact dermatitis.

18. The method according to claim 17, wherein the therapeutic amount of the composition is effective to mitigate miniaturization of hair follicles in androgen-sensitive areas of the scalp, and to re-launch stem cell activity and proliferation, without substantial scalp irritation.

19. The method according to claim 16, wherein the alopecia is a non-androgenetic alopecia.

20. The method according to claim 10, wherein the non-androgenic alopecia is selected from the group consisting of vertex alopecia, chemical alopecia, traction alopecia, traumatic alopecia and central centrifugal cicatricial alopecia (CCCA).

21. The method according to claim 17, wherein the subject is an Afro-American human subject.
